(19) United States
(12) Patent Application Publication
(10) Pub. No.: US 2014/0309523 A1
(43) Pub. Date: Oct. 16, 2014

(54) THREE-DIMENSIONAL EXTRACTION TRACKING FOR IMPLANT MODELING

(71) Applicant: Navigate Surgical Technologies, Inc., Vancouver (CA)

(72) Inventors: Ehud (Udi) Daon, North Vancouver (CA); Martin Gregory Beckett, Bowen Island (CA)

(73) Assignee: Navigate Surgical Technologies, Inc., Vancouver (CA)

(21) Appl. No.: 14/252,340

(22) Filed: Apr. 14, 2014

Related U.S. Application Data
(60) Provisional application No. 61/812,536, filed on Apr. 16, 2013.

(51) Int. Cl. A61B 19/00

(52) U.S. Cl. CPC A61B 19/50; A61B 19/5244; A61B 19/54; A61B 2019/502; A61B 2019/508; A61B 2017/00526
USPC 600/424; 29/407.01

(57) ABSTRACT

A method for making an insert for a bone structure located within a surgical site comprises operating on the bone structure with a materials removing tool to remove materials from the bone structure; tracking in three dimensions the location and orientation of the bone structure; tracking in three dimensions the location and orientation of the working tip of the materials removing tool; creating a software model of the materials volume removed from the bone structure; and making the insert based on model data within the software model. An associated system comprises a controller and a tracker for tracking a 3-D tracking marker rigidly attached to the bone structure and a 3-D tracking marker rigidly attached to the materials removing tool. A manufacturing device is used to manufacture the insert based on the model data from the controller.

### BACKGROUND OF THE INVENTION

1. Field of the Invention

The invention relates to location monitoring hardware and software systems. More specifically, the field of the invention is that of surgical equipment and software for monitoring surgical conditions and for deriving related surgical information in order to prepare surgical prostheses.

2. Description of the Related Art

Visual and other sensory systems for observing and monitoring surgical procedures are known in the art. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. For example, in one known system a carrier assembly bears at least one fiducial marker onto an attachment element in a precisely repeatable position with respect to a patient's jaw bone, employing the carrier assembly for providing registration between the fiducial marker and the patient's jaw bone and implanting the tooth implant by employing a tracking system which uses the registration to guide a drilling assembly. With this relatively new computer implemented technology, further improvements may further advance the effectiveness of surgical procedures.

In the area of prosthodontic surgery, existing teeth are often reshaped using a suitable drill in order to allow the attachment or insertion of various kinds of dental prostheses or implants. While techniques exist to model the prostheses to be located external to the existing or remaining teeth, implants or the implantable parts of prosthesis are typically reliant for their design on molding techniques employing materials such as sodium alginate, polyether and silicones. The molding techniques are inherently “blind”, in that the dentist or dental surgeon cannot see the area being molded during the molding process. Similar limitations apply to the more general field of bone surgery.

### SUMMARY OF THE INVENTION

In a first aspect, embodiments of the invention provide a method for making an insert into an insertion point on a bone structure located within a surgical site. The method comprises operating on the insertion point with a materials removing tool having a working tip to remove materials from the bone structure; tracking in three dimensions with a tracker a location and orientation of the bone structure; tracking in three dimensions with the tracker a location and orientation of the working tip of the materials removing tool; creating a software model of a volume originally occupied by the materials removed from the bone structure at the insertion point; and making the insert based on model data within the software model. The method may further comprise adding to the software model pre-existing structural information about a structure to be added to the insert.

The step of tracking in three dimensions the location and orientation of the bone structure may comprise tracking in three dimensions a location and orientation of a first three-dimensional tracking marker rigidly and removably attached to the bone structure proximate the insertion point. The first three-dimensional tracking marker may be rigidly and removably attached to the bone structure via a fiducial reference rigidly and removably attached to the bone structure proximate the insertion point. The fiducial reference may be marked or shaped for having its location and orientation determined from a scan of the surgical site.

The tracking in three dimensions the first three-dimensional tracking marker may comprise identifying the fiducial reference in a pre-existing scan of the surgical site; determining a three-dimensional location and orientation of the fiducial reference relative to the insertion point; and determining a three-dimensional location and orientation of the first three-dimensional tracking marker relative to the insertion point based on the three-dimensional location and orientation of the fiducial reference relative to the insertion point.

The step of tracking in three dimensions the location and orientation of the working tip of the materials removing tool may comprise tracking in three dimensions the location and orientation of a second three-dimensional tracking marker rigidly attached to the material removing tool with a predetermined relative location and orientation relative to the working tip.

The step of creating a software model of the volume originally occupied by the materials removed from the bone structure at the insertion point may comprise deriving bone structure tracking information from the tracking of the bone structure; deriving working tip tracking information from the tracking of the working tip; and deriving a three-dimensional materials removal map based on an overlap of the bone structure tracking information and the working tip tracking information.

The step of making the insert may comprise extracting model data from the software model; providing the model data to a manufacturing device; and manufacturing the insert based on the model data. The model data may be provided directly to the manufacturing device or may first be stored on a storage medium and then provided to a manufacturing device.

In a further aspect, embodiments of the invention provide a system for making an insert into an insertion point on a bone structure located within a surgical site, the system comprising a tracker having a field of view including the surgical site and arranged for obtaining image information about the surgical site; a first three-dimensional tracking marker rigidly and removably attached to the bone structure proximate the insertion point and within the field of view; a materials removal tool having a working tip and a rigidly attached second three-dimensional tracking marker and within the field of view; and a controller comprising at least one processor and at least one memory, the memory including scan data of the surgical site, and software configured, when executed by the at least one processor, to create based on the scan data and on the image information a software model of a volume originally occupied by materials removed by the materials removal tool from the bone structure at the insertion point.

The first three-dimensional tracking marker may be rigidly and removably attached to the bone structure via a fiducial reference rigidly and removably attached to the bone structure proximate the insertion point and the fiducial reference may be marked or shaped for having its location and orientation determined from the scan data. In other embodiments the first three-dimensional tracking marker may be a fiducial reference rigidly and removably attached to the bone structure proximate the insertion point and the first tracking marker may be marked or shaped for having its location and orientation determined from the scan data.

The software may further comprise a first set of executable instructions stored in the memory and executable by the processor, the first set of executable instructions comprising a second set of instructions for creating the software model based on the scan data and the image information; and a third set of instructions for extracting model data from the software model, the model data configured to provide a manufacturing device with manufacturing information relating to the insert according to the software model.

The second set of instructions may comprise instructions for obtaining the scan data; instructions for obtaining the image information from the tracker; instructions for obtaining bone structure tracking information from the image information; instructions for obtaining working tip tracking information from the image information; instructions for obtaining a three-dimensional materials removal map based on an overlap of the bone structure tracking information and the working tip tracking information; and instructions for establishing a model exterior shape for the insert based on the three-dimensional materials removal map.

The system may further comprise a manufacturing device in communication with the controller, the manufacturing device configured to manufacture the insert based on the model data according to the software model.

In another aspect of the invention, embodiments of the invention involve a system for making an insert to be inserted into an insertion point on a bone structure located within a surgical site, the system comprising a tracker having a field of view capable of including the surgical site and capable of being arranged for obtaining image information about the surgical site. The system also includes a first three-dimensional tracking marker capable of being rigidly and removably attached to the bone structure proximate the insertion point and within the field of view. Further included in the system is a materials removal tool having a working tip and a rigidly attached second three-dimensional tracking marker and capable of being positioned within the field of view. The system controller comprises at least one processor and at least one memory, the at least one memory including scan data of the surgical site, and software configured, when executed by the at least one processor, to create based on the scan data and on the image information a software model of a volume originally occupied by materials removed by the materials removal tool from the bone structure at the insertion point.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-J are drawings of hardware components of the surgical monitoring system according to embodiments of the invention.

FIGS. 4A-C is a flow chart diagram illustrating one embodiment of the registering method of the present invention.

FIG. 5 is a drawing of a dental fiducial key with a tracking pole and a dental drill according to one embodiment of the present invention.

FIG. 6 is a drawing of an endoscopic surgical site showing the fiducial key, endoscope, and biopsy needle according to another embodiment of the invention.

FIG. 7 is an extension and elaboration of FIG. 5 showing drawing of a system for making an insert into an insertion point on a bone structure located within a surgical site.

FIG. 8 shows a schematic a flow diagram for a method to prepare a surgical insert for bone surgery

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipments through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (“CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.) , and Windows Phone 7. “Mobile Apps” refers to software programs written for execution with Mobile Software.

The terms “scan,” “fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient. The term “fiducial reference” or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. In some embodiments, the tracker may be a non-stereo optical tracker, for example a camera. The camera may, for example, operate in the visible or near-infrared range. The term “image information” is used in the present specification to describe information obtained by the tracker, whether optical or otherwise, and usable for determining the location of the markers and their orientation and movement continually in ‘real time’ during a procedure.

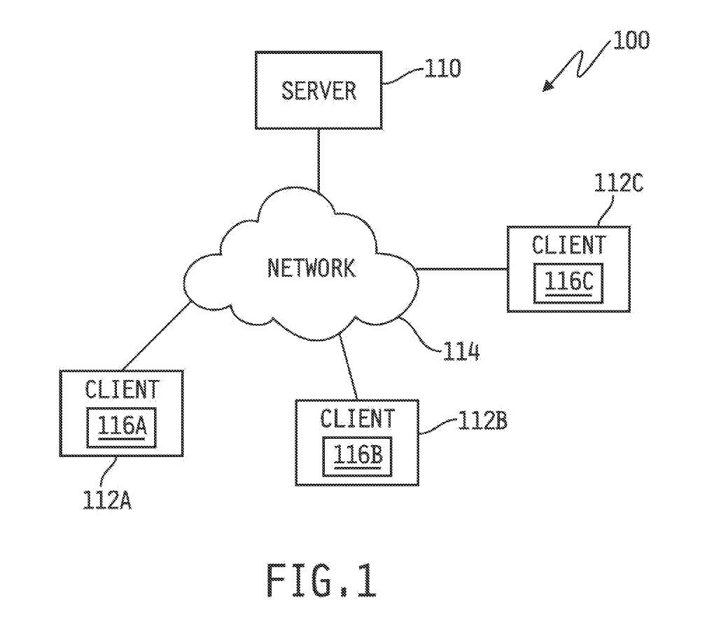

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

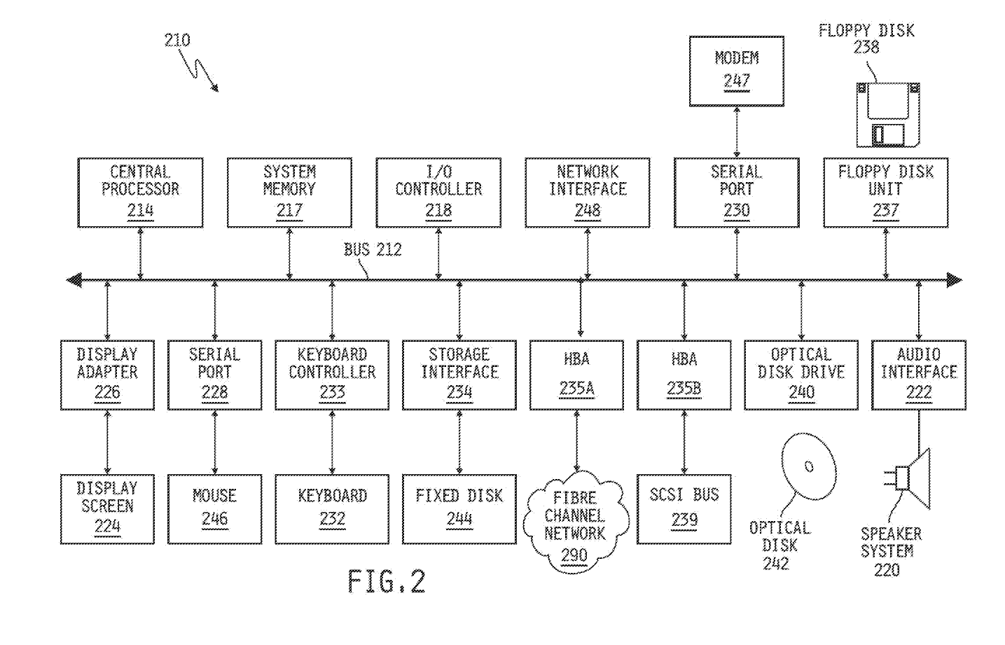

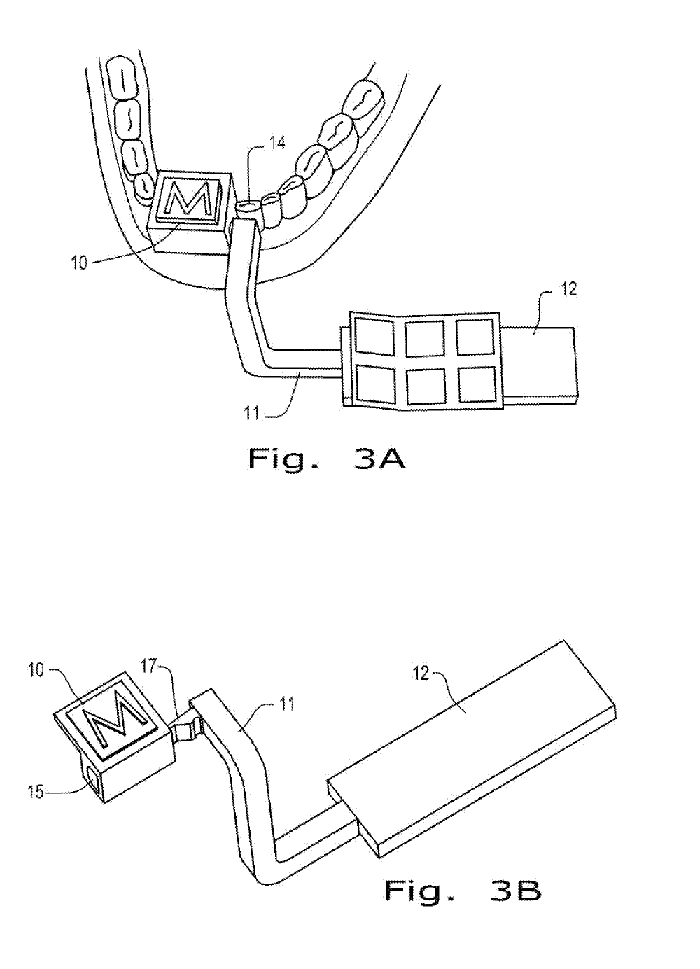

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

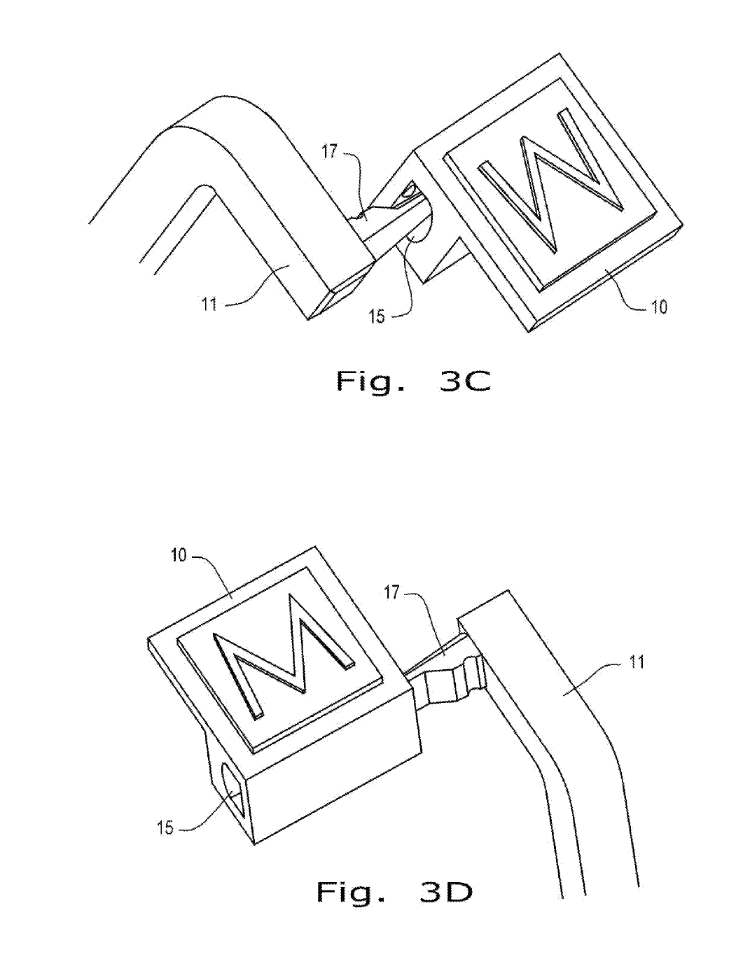

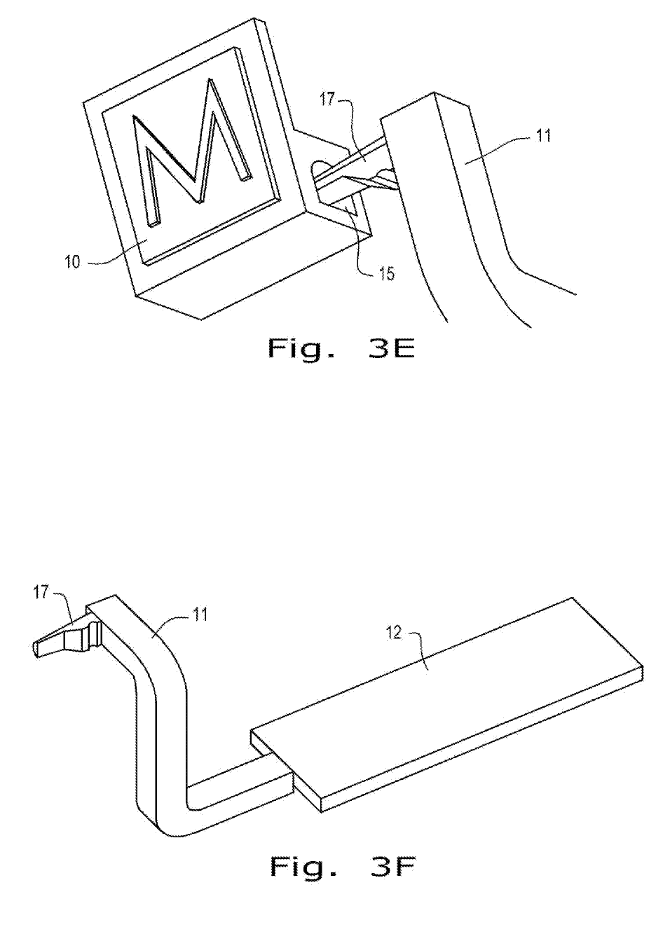

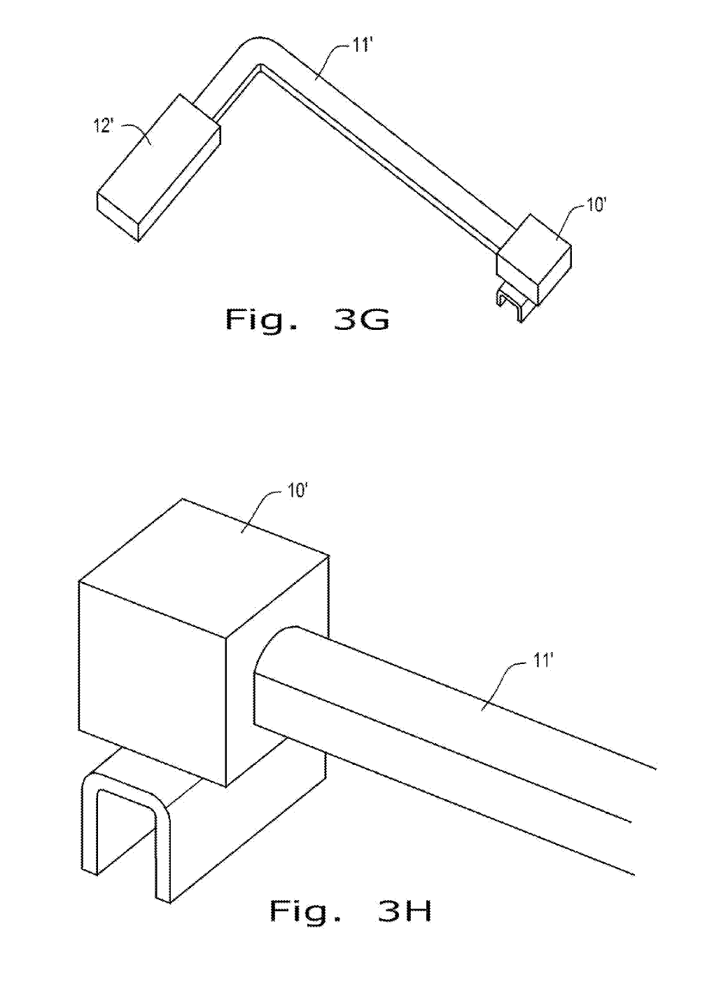

Many other devices or subsystems (not shown) may be connected in a similar manner (e.g., document scanners, digital cameras and so on), including the hardware components of FIGS. 3A-I, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

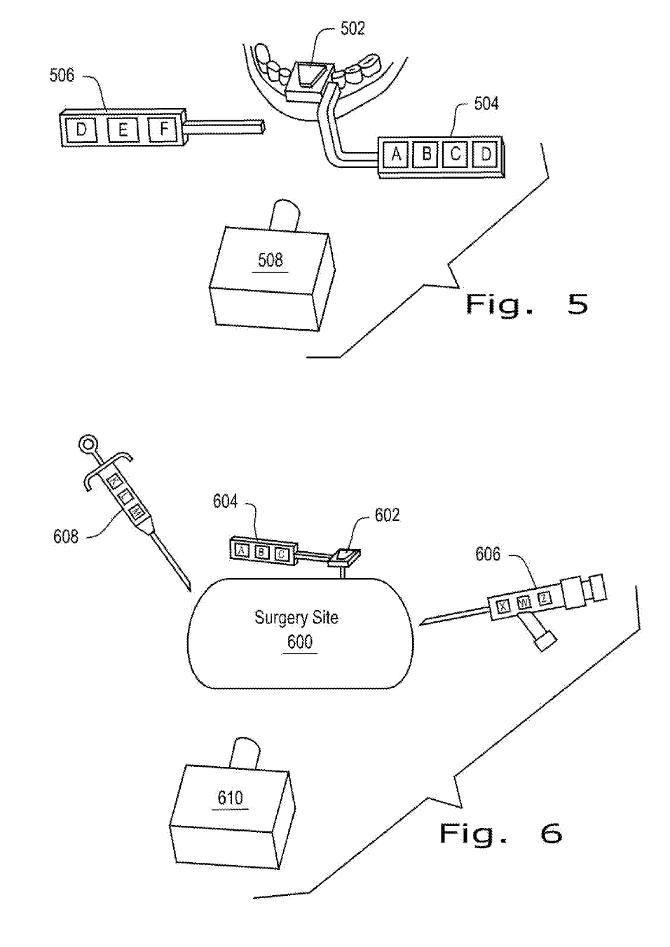

Embodiments of the present invention relate to a surgical hardware and software monitoring system and method which allows for surgical planning while the patient is available for surgery, for example while the patient is being prepared for surgery so that the system may model the surgical site. The system uses a particularly configured piece of hardware, represented as single fiducial key 10 in FIG. 3A, to orient tracking marker 12 of the monitoring system with regard to the critical area of the surgery. Single fiducial key 10 is attached to a location near the intended surgical area, in the exemplary embodiment of the dental surgical area of FIG. 3A, fiducial key 10 is attached to a dental splint 14. Tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference. The tracker may be a non-stereo optical tracker. For example a dental surgery, the dental traking marker 14 may be used to securely locate the fiducial 10 near the surgical area. Single fiducial key 10 may be used as a point of reference, or a fiducial, for the further image processing of data acquired from tracking marker 12 by the tracker.

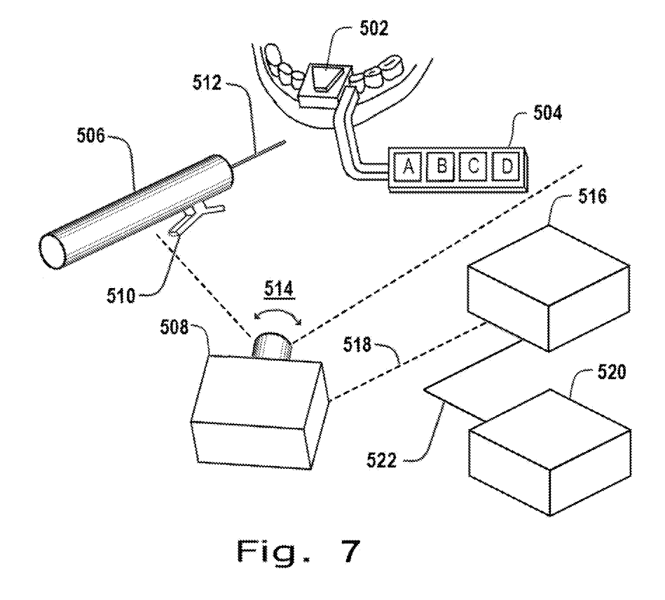

In other embodiments additional tracking markers 12 may be attached to items independent of the fiducial key 10 and any of its associated tracking poles 11 or tracking markers 12. This allows the independent items to be tracked by the tracker. Further embodiments of such additional tracking markers are discussed in detail below at the hand of FIGS. 6 and 7.

In a further embodiment at least one of the items or instruments near the surgical site may optionally have a tracker attached to function as tracker for the monitoring system of the invention and to thereby sense the orientation and the position of the tracking marker 12 and of any other additional tracking markers relative to the scan data of the surgical area. By way of example, the tracker attached to an instrument may be a miniature digital camera and it may be attached, for example, to a dentist's drill. Any other markers to be tracked by the tracker attached to the item or instrument must be within the field of view of the tracker.

Using the dental surgery example, the patient is scanned to obtain an initial scan of the surgical site. The particular configuration of single fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent as a recognizable identifying symbol when scanned. In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker display the arrangement in real time on a suitable display, for example display 224 of FIG. 2.

In one embodiment, the computer system has a predetermined knowledge of the physical configuration of fiducial key 10 and examines slices/sections of the scan to locate fiducial key 10. Locating of fiducial key 10 may be on the basis of its distinct shape, or on the basis of distinctive identifying and orienting markings upon the fiducial key or on attachments to the fiducial key 10 as tracking marker 12. Fiducial key 10 may be rendered distinctly visible in the scans through higher imaging contrast by the employ of radio-opaque materials or high-density materials in the construction of the fiducial key 10. In other embodiments the material of the distinctive identifying and orienting markings may be created using suitable high density or radio-opaque inks or materials.

Once single fiducial key 10 is identified, the location and orientation of the fiducial key 10 is determined from the scan segments, and a point within fiducial key 10 is assigned as the center of the coordinate system. The point so chosen may be chosen arbitrarily, or the choice may be based on some useful criterion. A model is then derived in the form of a transformation matrix to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. For example, as particularly shown in FIG. 3J, single fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment.

It is further possible to reorient the tracking pole during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of the tracking pole may trigger a re-registration of the tracking pole with relation to the coordinate system, so that the locations may be accordingly adjusted. Such a re-registration may be automatically initiated when, for example in the case of the dental surgery embodiment, tracking pole 11 with its attached tracking marker 12 are removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas.

In a further embodiment of the system utilizing the invention, a surgical instrument or implement, herein termed a “hand piece” (see FIGS. 5, 6 and 7), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, or an alarm sound. Further, target boundary conditions may be set up to indicate the desired surgical area, so that when the trajectory of the hand piece is trending outside the target area an indication may appear on screen or an alarm sound indicating that the hand piece is deviating from its desired path.

An alternative embodiment of some hardware components are shown in FIGS. 3G-I. Single fiducial key 10′ has connection elements with suitable connecting portions to allow a tracking pole 11′ to position a tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key must be suitable for connection to a plastic splint and suitable for connection to a tracking pole. In the surgical example the materials of the fiducial key may be suitable for attachment to the skin or other particular tissue of a patient.

The tracking markers are clearly identified by employing, for example without limitation, high contrast pattern engraving. The materials of the tracking markers are chosen to be capable of resisting damage in autoclave processes and are compatible with rigid, repeatable, and quick connection to a connector structure. The tracking markers and associated tracking poles have the ability to be accommodated at different locations for different surgery locations, and, like the fiducial keys, they should also be relatively lightweight as they will often be resting on or against the patient. The tracking poles must similarly be compatible with autoclave processes and have connectors of a form shared among tracking poles.

The tracker employed in tracking the fiducial keys, tracking poles and tracking markers should be capable of tracking with suitable accuracy objects of a size of the order of 1.5 square centimeters. The tracker may be, by way of example without limitation, a stereo camera or stereo camera pair. While the tracker is generally connected by wire to a computing device to read the sensory input, it may optionally have wireless connectivity to transmit the sensory data to a computing device.

In embodiments that additionally employ a trackable piece of instrumentation, such as a hand piece, tracking markers attached to such a trackable piece of instrumentation may also be light-weight; capable of operating in a 3 object array with 90 degrees relationship; optionally having a high contrast pattern engraving and a rigid, quick mounting mechanism to a standard hand piece. In other embodiments the tracking markers are monolithically integrated with a rigid positioning and orienting portion of the hand piece, as described in more detail at the hand of FIGS. 6 and 7.

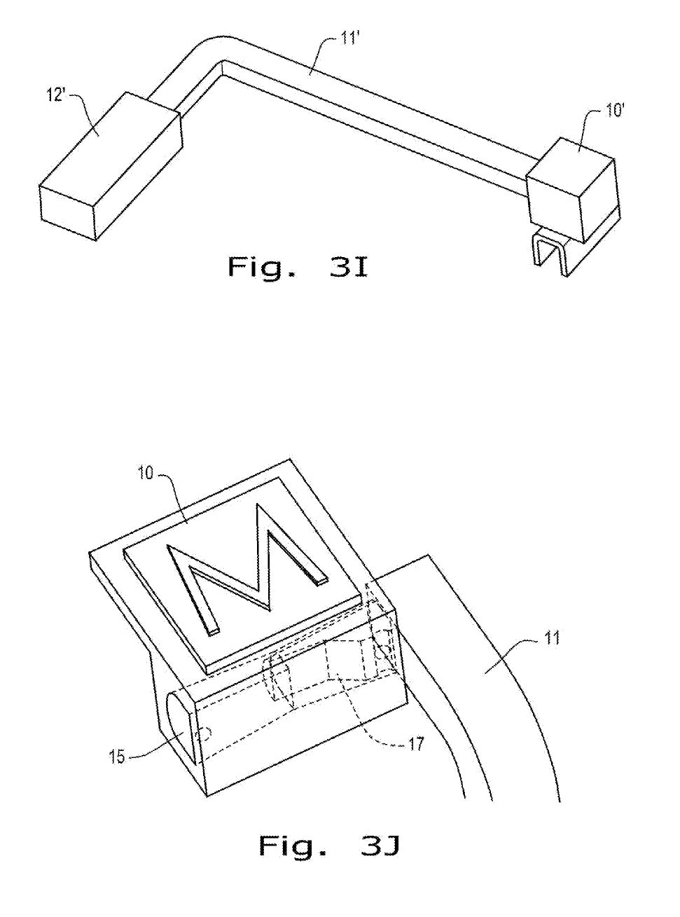

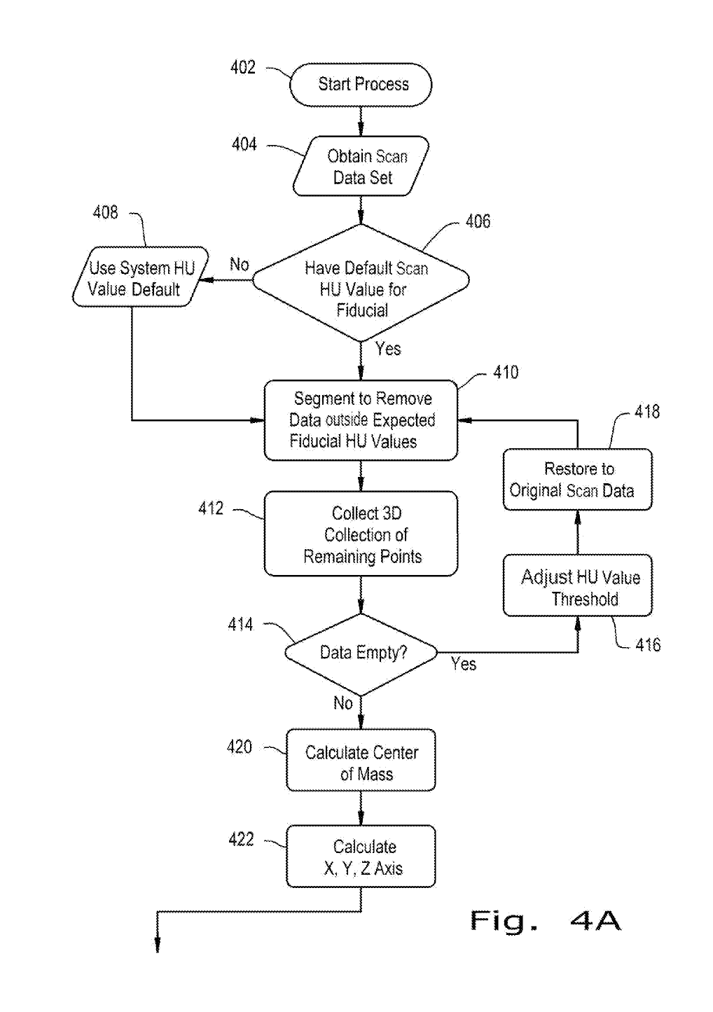

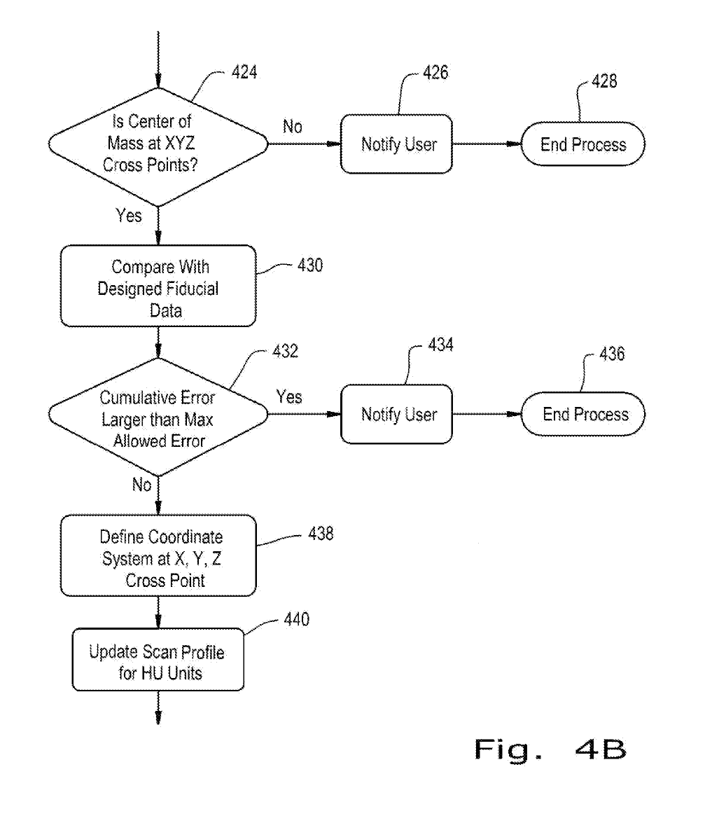

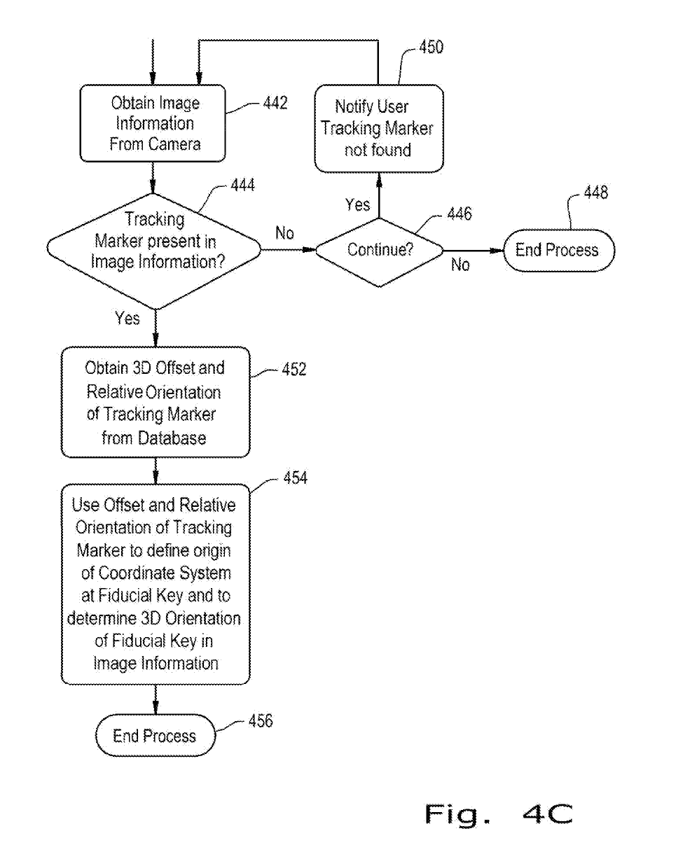

In another aspect of the invention there is presented an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains a scan data set [404] from, for example, a CT scanner and checks for a default CT scan Hounsfield unit (HU) value [at 406] for the fiducial which may or may not have been provided with the scan based on a knowledge of the fiducial and the particular scanner model, and if such a threshold value is not present, then a generalized predetermined default value is employed [408]. Next the data is processed by removing scan segments with Hounsfield data values outside expected values associated with the fiducial key values [at 410], following the collection of the remaining points [at 412]. If the data is empty [at 414], the CT value threshold is adjusted [at 416], the original value restored [at 418], and the segmenting processing scan segments continues [at 410]. Otherwise, with the existing data a center of mass is calculated [at 420], along with calculating the X, Y, and Z axes [at 422]. If the center of mass is not at the cross point of the XYZ axes [at 424], then the user is notified [at 426] and the process stopped [at 428]. If the center of mass is at the XYZ cross point then the data points are compared with the designed fiducial data [430]. If the cumulative error is larger than the maximum allowed error [432] then the user is notified [at 434] and the process ends [at 436]. If not, then the coordinate system is defined at the XYZ cross point [at 438], and the scan profile is updated for the HU units [at 440].

Turning now to FIG. 4C, image information is obtained from the tracker, being a suitable camera or other sensor [442]. The image information is analyzed to determine whether a tracking marker is present in the image information [444]. If not, then the user is queried [446] as to whether the process should continue or not. If not, then the process is ended [448]. If the process is to continue, then the user can be notified that no tracking marker has been found in the image information [450], and the process returns to obtaining image information [442]. If a tracking marker has been found based on the image information, or one has been attached by the user upon the above notification [450], the offset and relative orientation of the tracking marker to the fiducial reference is obtained from a suitable database [452]. The term “database” is used in this specification to describe any source, amount or arrangement of such information, whether organized into a formal multi-element or multi-dimensional database or not. A single data set comprising offset value and relative orientation may suffice in a simple implementation of this embodiment of the invention and may be provided, for example, by the user or may be within a memory unit of the controller or in a separate database or memory.

The offset and relative orientation of the tracking marker is used to define the origin of a coordinate system at the fiducial reference and to determine the three-dimensional orientation of the fiducial reference based on the image information [454] and the registration process ends [458]. In order to monitor the location and orientation of the fiducial reference in real time, the process may be looped back from step [454] to obtain new image information from the camera [442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing tracking markers in the proximity of the surgical site. Other registration systems are also contemplated, for example using current other sensory data rather than the predetermined offset, or having a fiducial with a transmission capacity.

One example of an embodiment of the invention is shown in FIG. 5. In addition to fiducial key 502 mounted at a predetermined tooth and having a rigidly mounted tracking marker 504, an additional instrument or implement 506, for example a hand piece which may be a dental drill, may be observed by a camera 508 serving as tracker of the monitoring system.

Another example of an embodiment of the invention is shown in FIG. 6. Surgery site 600, for example a human stomach or chest, may have fiducial key 602 fixed to a predetermined position to support tracking marker 604. Other apparatus with suitable tracking markers may be in use in the process of the surgery at surgery site 600. By way of non-limiting example, endoscope 606 may have a further tracking marker, and biopsy needle 608 may also be present bearing a tracking marker at surgery site 600. Sensor 610, serving as tracker for the system, may be for example a camera, infrared sensing device, or RADAR. In particular, the tracker may be a two-dimensional imaging tracker that produces a two dimensional image of the surgery site 600 for use as image information for the purposes of embodiments of the invention, including two dimensional image information of any tracking markers in the field of view of the tracker. More particularly, tracker 610 may be a non-stereo optical tracker. Surgery site 600, endoscope 606, biopsy needle 608, fiducial key 602 and tracking marker 604 may all be in the field of view of tracker 610.

A further aspect of the present invention is described in the embodiment of FIG. 7, which is a schematic elaboration and extension of FIG. 5. Dental drill 506 with drill bit 512 bears tracking marker 510. The exact location and orientation of the tip of drill bit 512 relative to tracking marker 510 is known and available to controller 516. Controller 516 may be for example processor 214 and memory 217 of computer 210 of FIG. 2. Fiducial reference 502 is mounted at a predetermined position within the surgical site and has rigidly mounted tracking marker 504. Tracking marker 504 and tracking marker 510 are in field of view 514 of tracker 508. Tracking markers 510 and 504 are tracked within field of view 514 by tracker 508, which may is some embodiments be a suitable camera. The camera may be a measuring camera. The camera may be equipped with a telecentric lens. Tracker 508 supplies image information about the surgical site depicted schematically in FIG. 7 over interface 518 to controller 516. While interface 518 is generally a wired interface, it may be without limitation a wireless interface.

Controller 516 has prior scan data of the surgical site containing fiducial reference 502. The three dimensional spatial relationship between fiducial reference 502 and tracking marker 504 is fixed and known. Tracker 508 may therefore track the location and orientation of the tip of drill bit 512 in three dimensions with respect to the surgical site and, more particularly, with respect to fiducial reference 502. Since the prior scan data provides the location and orientation of a target tooth relative to fiducial reference 502, controller 516 has all the spatial information to calculate the position and orientation of the tip of drill bit 512 relative to the target tooth.

When drill bit 512 physically interacts with the target tooth and removes material from that tooth, the image information supplied to controller 516 allows controller 516 to compute the three-dimensional shape of the material removed and to create a model of the volume originally occupied by the removed material. Software of controller 516 then proceeds to generate a manufacturing program based on the model and executable by manufacturing device 520 so that the manufacturing device 520 manufactures an article according to the model, for example by creating a part program for a Computer Numerical Controlled (CNC) machine tool or creating a Computer Aided Design (CAD) rendered to a Standard Tessellation Language (STL) file format for a three dimensional printer. That model is saved into an appropriate three-dimensional file format using either a default or user chosen file format.

If controller 516 is locally connected to manufacturing device 520 via wired interface 522 then the saved file containing the three dimensional manufacturing pattern may be directly sent to manufacturing device 520. Alternatively, the user may select a destination machine for the saved file, which may optionally be a remote machine wherein the saved file is electronically transmitted, e.g., by e-mail or file transfer protocol (FTP) over a network such as the Internet. In a further alternative, after the selection of a destination machine the system may store the saved file with the three dimensional manufacturing pattern to a computer readable media to be loaded into an appropriate manufacturing machine at a later time.

If, for example, a new crown is to be fashioned, the model of the volume originally occupied by the removed material may be combined in software by controller 516 with an available model of the crown and the combined three-dimensional structure may be manufactured by manufacturing device 520. In one embodiment, software of controller 516 determines the exact shape of the removed material. In another embodiment, software of controller 516 uses the shape of the removed material and other parameters to select an appropriate model for a replacement part which may be larger or smaller than the removed material.

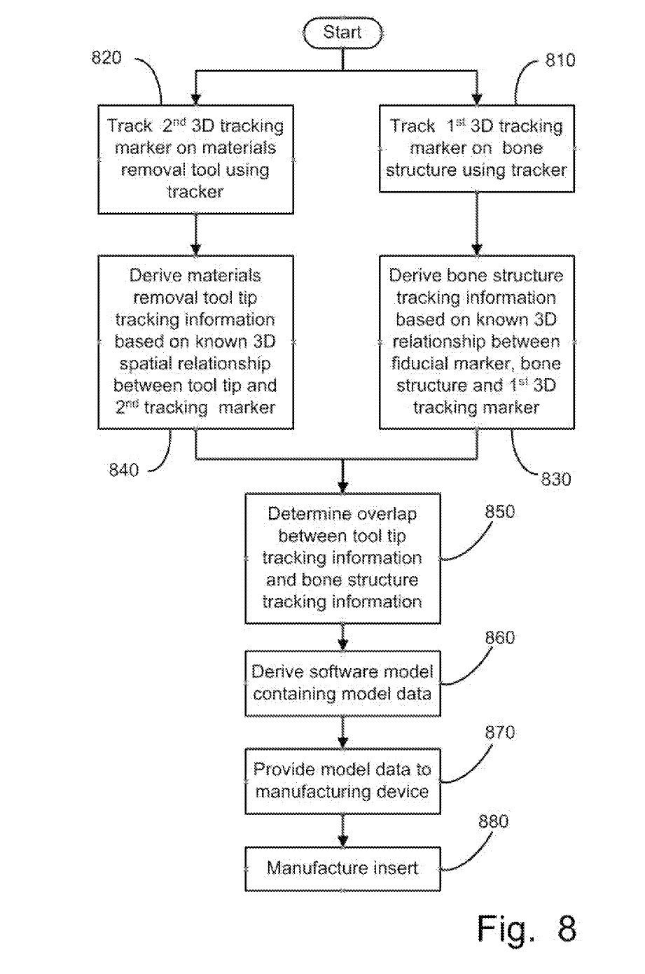

In another aspect, embodiments of the invention provide a method for making an insert into an insertion point on a bone structure located within a surgical site as shown in the flow diagram of FIG. 8. The method comprises operating on the insertion point with a materials removing tool having a working tip to remove materials from the bone structure; tracking [830] in three dimensions with a tracker a location and orientation of the bone structure; tracking [840] in three dimensions with the tracker a location and orientation of the working tip of the materials removing tool; creating a software model [860] of a volume originally occupied by the materials removed from the bone structure at the insertion point; and making the insert based on model data within the software model. The method may further comprise adding to the software model pre-existing structural information about a structure to be added to the insert. One non-limiting example is the addition of a prosthetic tooth crown to the insert.

Step [830] of tracking in three dimensions the location and orientation of the bone structure may comprise tracking [810] in three dimensions a location and orientation of a first three-dimensional tracking marker rigidly and removably attached to the bone structure proximate the insertion point. The first three-dimensional tracking marker may be rigidly and removably attached to the bone structure via a fiducial reference rigidly and removably attached to the bone structure proximate the insertion point. The fiducial reference may be marked or shaped for having its location and orientation determined from a scan of the surgical site.

Step [810] of tracking in three dimensions of the first three-dimensional tracking marker may comprise identifying the fiducial reference in a pre-existing scan of the surgical site; determining a three-dimensional location and orientation of the fiducial reference relative to the insertion point; and determining a three-dimensional location and orientation of the first three-dimensional tracking marker relative to the insertion point based on the three-dimensional location and orientation of the fiducial reference relative to the insertion point.

Step [840] of tracking in three dimensions the location and orientation of the working tip of the materials removing tool may comprise tracking [820] in three dimensions the location and orientation of a second three-dimensional tracking marker rigidly attached to the materials removing tool with a predetermined relative location and orientation relative to the working tip.

Step [860] of creating a software model of the volume originally occupied by the materials removed from the bone structure at the insertion point may comprise deriving bone structure tracking information from the tracking of the bone structure; deriving working tip tracking information from the tracking of the working tip; and deriving [850] a three-dimensional materials removal map based on an overlap of the bone structure tracking information and the working tip tracking information.

The step of making the insert may comprise extracting model data from the software model; providing [870] the model data to a manufacturing device; and manufacturing [880] the insert based on the model data. The model data may be provided directly to the manufacturing device or may first be stored on a storage medium and then provided to a manufacturing device. The manufacturing device may be a remotely located manufacturing device.

Embodiments of the invention provide an apparatus and method for the manufacture of inserts, implants and prostheses. It has been described in the context of embodiments relating to an example based on dental surgery, but is not limited to that field. It may equally well be employed in other forms of bone surgery. The apparatus and method are applicable to cases where the three-dimensional shape of materials removed from an existing three-dimensional shape needs to be tracked and where, in some instances, it may need to be manufactured as an implant or in which an insert has to be made into the object from which materials have been removed in order to serve as a means of attachment.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for making an insert into an insertion point on a bone structure located within a surgical site, the method comprising:
operating on the insertion point with a materials removing tool having a working tip to remove materials from the bone structure;
tracking in three dimensions with a tracker a location and orientation of the bone structure;
tracking in three dimensions with the tracker a location and orientation of the working tip of the materials removing tool;
creating a software model of a volume originally occupied by the materials removed from the bone structure at the insertion point; and
making the insert based on model data within the software model.

2. The method of claim 1, wherein the step of tracking in three dimensions the location and orientation of the bone structure comprises tracking in three dimensions a location and orientation of a first three-dimensional tracking marker rigidly and removably attached to the bone structure proximate the insertion point.

3. The method of claim 2, wherein the first three-dimensional tracking marker is rigidly and removably attached to the bone structure via a fiducial reference rigidly and removably attached to the bone structure proximate the insertion point and wherein the fiducial reference is at least one of marked and shaped for having its location and orientation determined from a scan of the surgical site.

4. The method of claim 3, wherein tracking in three dimensions the first three-dimensional tracking marker comprises
identifying the fiducial reference in a pre-existing scan of the surgical site;
determining a three-dimensional location and orientation of the fiducial reference relative to the insertion point; and
determining a three-dimensional location and orientation of the first three-dimensional tracking marker relative to the insertion point based on the three-dimensional location and orientation of the fiducial reference relative to the insertion point

5. The method of claim 1, wherein the step of tracking in three dimensions the location and orientation of the working tip of the materials removing tool comprises tracking in three dimensions the location and orientation of a second three-dimensional tracking marker rigidly attached to the material removing tool with a predetermined relative location and orientation relative to the working tip.

6. The method of claim 1, wherein the step of creating a software model of the volume originally occupied by the materials removed from the bone structure at the insertion point comprises:
deriving bone structure tracking information from the tracking of the bone structure;
deriving working tip tracking information from the tracking of the working tip; and
deriving a three-dimensional materials removal map based on an overlap of the bone structure tracking information and the working tip tracking information.

7. The method of claim 1, wherein the step of making the insert comprises extracting model data from the software model;
providing the model data to a manufacturing device; and
manufacturing the insert based on the model data.

8. The method of claim 7, wherein the model data is one of provided directly to the manufacturing device; and
first stored on a storage medium and then provided to a manufacturing device.

9. The method of claim 7, further comprising adding to the software model pre-existing structural information about a structure to be added to the insert.

10. A system for making an insert to be inserted into an insertion point on a bone structure located within a surgical site, the system comprising:
a tracker having a field of view capable of including the surgical site and capable of being arranged for obtaining image information about the surgical site;
a first three-dimensional tracking marker capable of being rigidly and removably attached to the bone structure proximate the insertion point and within the field of view;
a materials removal tool having a working tip and a rigidly attached second three-dimensional tracking marker and capable of being positioned within the field of view; and
a controller comprising at least one processor and at least one memory, the at least one memory including scan data of the surgical site, and software configured, when executed by the at least one processor, to create based on the scan data and on the image information a software model of a volume originally occupied by materials removed by the materials removal tool from the bone structure at the insertion point.

11. The system of claim 10, wherein the software further comprises a first set of executable instructions stored in the at least one memory and executable by the at least one processor, the first set of executable instructions comprising a second set of instructions for creating the software model based on the scan data and the image information; and a third set of instructions for extracting model data from the software model, the model data configured to provide a manufacturing device with manufacturing information relating to the insert according to the software model.

12. The system of claim 11, wherein the second set of instructions comprises instructions for obtaining the scan data;
instructions for obtaining the image information from the tracker;
instructions for obtaining bone structure tracking information from the image information;
instructions for obtaining working tip tracking information from the image information;
instructions for obtaining a three-dimensional materials removal map based on an overlap of the bone structure tracking information and the working tip tracking information; and
instructions for establishing a model exterior shape for the insert based on the three-dimensional materials removal map.

13. The system of claim 11, further comprising a manufacturing device in communication with the controller, the manufacturing device configured to manufacture the insert based on the model data according to the software model.

14. The system of claim 10, wherein the first three-dimensional tracking marker is rigidly and removably attached to the bone structure via a fiducial reference rigidly and removably attached to the bone structure proximate the insertion point and wherein the fiducial reference is at least one of marked and shaped for having its location and orientation determined from the scan data.

15. The system of claim 10, wherein the first three-dimensional tracking marker is a fiducial reference rigidly and removably attached to the bone structure proximate the insertion point and wherein the first tracking marker is at least one of marked and shaped for having its location and orientation determined from the scan data.

16. A method for making an insert to be inserted into an insertion point on a bone structure, the method comprising:
tracking in three dimensions with a tracker a location and orientation of the bone structure;
tracking in three dimensions with the tracker a location and orientation of a working tip of a materials removing tool suitable for operating on the insertion point and for removing materials from the bone structure;
creating a software model of a volume originally occupied by the materials removed from the bone structure at the insertion point; and
making the insert based on model data within the software model.

17. The method of claim 16, wherein the step of tracking in three dimensions the location and orientation of the bone structure comprises tracking in three dimensions a location and orientation of a first three-dimensional tracking marker rigidly and removably attached to the bone structure proximate the insertion point, wherein the first three-dimensional tracking marker is rigidly and removably attached to the bone structure via a fiducial reference rigidly and removably attached to the bone structure proximate the insertion point and wherein the fiducial reference is at least one of marked and shaped for having its location and orientation determined from a scan of the surgical site, wherein tracking in three dimensions the first three-dimensional tracking marker comprises:
identifying the fiducial reference in a pre-existing scan of the surgical site;
determining a three-dimensional location and orientation of the fiducial reference relative to the insertion point; and
determining a three-dimensional location and orientation of the first three-dimensional tracking marker relative to the insertion point based on the three-dimensional location and orientation of the fiducial reference relative to the insertion point

18. The method of claim 16, wherein the step of tracking in three dimensions the location and orientation of the working tip of the materials removing tool comprises tracking in three dimensions the location and orientation of a second three-dimensional tracking marker rigidly attached to the material removing tool with a predetermined relative location and orientation relative to the working tip.

19. The method of claim 16, wherein the step of creating a software model of the volume originally occupied by the materials removed from the bone structure at the insertion point comprises:
deriving bone structure tracking information from the tracking of the bone structure;
deriving working tip tracking information from the tracking of the working tip; and
deriving a three-dimensional materials removal map based on an overlap of the bone structure tracking information and the working tip tracking information.

20. The method of claim 16, wherein the step of making the insert comprises:
extracting model data from the software model;
providing the model data to a manufacturing device; and
manufacturing the insert based on the model data.
